(19) United States
(12) Patent Application Publication
(10) Pub. No.: US 2009/0248048 A1
(43) Pub. Date: Oct. 1, 2009

(54) PROSTHETIC FOR TISSUE REINFORCEMENT

(75) Inventor: Michael Milbocker, Holliston, MA (US)

Correspondence Address: RISSMAN HENDRICKS & OLIVERIO, LLP, 100 Cambridge Street, Suite 2101, BOSTON, MA, 02114, US

(73) Assignee: Promethean Surgical Devices, LLC, Woburn, MA (US)

(21) Appl. No.: 12/482,495

(22) Filed: Jun. 11, 2009

Related U.S. Application Data
(63) Continuation of application No. 11/406,553, filed on Apr. 19, 2006, now abandoned.
(60) Provisional application No. 60/673,208, filed on Apr. 19, 2005.

(51) Int. Cl. A61B 17/08; A61F 2/02

(52) U.S. Cl. 606/151; 523/113

(57) ABSTRACT

A process for the manufacture of a prosthetic sheet with improved tissue healing characteristics useful in reinforcing tissue defects is disclosed. Generally the prosthetic may be comprised of any material that does not promote fibrosis and inflammation. In particular, the prosthetic may be comprised of non-absorbable hydrogel reinforced with fiber, so that the fiber reinforcement is encapsulated and shielded from interaction with tissue. The prosthetic may contain pores that pass through it to encourage tissue through-growth. These pores may be made by removing material from a sheet of reinforced hydrogel or the reinforcement means may contain a porosity around which the hydrogel is formed and the porosity is maintained.

This application claims the benefit of the priority of U.S. Provisional application Ser. 60/673,208, filed Apr. 19, 2005, which is hereby incorporated by reference in its entirety.

### FIELD OF THE INVENTION

This invention relates generally to permanent tissue reinforcing prosthetics that are implanted in the body. The invention also relates to methods of manufacturing such prosthetics. Additionally, the invention relates to topologies of reinforcing prosthetics meant to achieve certain healing dynamics and long-term biocompatibility. In particular, the coated reinforcements of the invention are resistant to puckering during healing, and to persistent microbial colonization.

### BACKGROUND OF THE INVENTION

It is an established practice in the surgical field to use mesh, absorbable and non-absorbable, to repair defects in tissue. In hernia and prolapse repair, Prolene and Mersilene brand meshes, manufactured and sold by Ethicon, Inc., Somerville, N.J., are sometimes used. Marlex brand mesh, an Atlas polypropylene monofilament knit, has also been used for tissue repair. Polypropylene is used because it promotes a fibroblastic response, but this response is only important for ensuring mesh fixation. Knitted and woven fabrics constructed from a variety of synthetic fibers have been used in surgical repair. These are described in at least U.S. Pat. Nos. 3,054,406; 3,124,136; 4,193,137; 4,347,847; 4,452,245; 4,520,821; 4,633,873; 4,652,264; 4,655,221; 4,838,884; 5,002,551; and European Patent Application No. 334,046.

Polypropylene meshes have generally been preferred, but in addition to polypropylene, mesh can also be comprised of a polyester. However, the success of this mesh as a long-term tissue reinforcement has been questioned because of its alleged instability in the body. Polytetrafluoroethylene (PTFE) has also been used in a sheet format as a tissue reinforcement, but these sheets tend to promote infection, even in a micro-porous or expanded format.

Hydrogel-based tissue reinforcement prosthetics are not currently used in surgical repair, primarily because such prosthetics are expected to provide permanent tissue support and most hydrogels are either absorbable or possess little tensile strength.

Our copending published application US 2002-0049503 discloses a tissue reinforcement prosthetic comprised of a hydrated hydrogel. Our copending application U.S. Ser. No. 11/010,629 discloses a tissue reinforcement prosthetic comprised of a mesh coated with a hydrogel. Each of these applications is incorporated by reference in its entirety.

U.S. Pat. No. 4,373,009 describes a method of coating polymeric substrates with polyurethane prepolymers containing free isocyanate groups and further coating this prepared substrate with a second coat of water-soluble copolymers of unsaturated monomers containing in the backbone of these copolymers at least some isocyanate-reactive monomers.

U.S. Pat. Nos. 4,459,317 and 4,487,808 disclose a process for coating a polymer substrate with an isocyanate solution containing at least two unreacted isocyanate groups per molecule, and, optionally, a polymer. This preparation is further coated with a high molecular weight polyethylene oxide, such that the two coatings form a hydrophilic polyethylene oxide-polyurea interpolymer with a low coefficient of friction. Methods for using solvents to apply a base coat of low molecular weight aromatic or aliphatic polyisocyanates followed by evaporation of the solvent and then application of a second coat of a high molecular weight polyethylene oxide polymer, also dissolved in an organic solvent, are disclosed.

U.S. Pat. No. 4,990,357 discloses compositions of chain-extended hydrophilic thermoplastic polyetherurethane polymers with hydrophilic high molecular weight non-urethane polymers. U.S. Pat. Nos. 5,077,352 and 5,179,174 describe methods for making coatings applied to a variety of substrates by crosslinking polyurethanes in the presence of polyethylene oxide polymers at high temperature. U.S. Pat. No. 6,265,016 describes medical prosthetics coated with a bonding layer of hydrogel. The bonds are accomplished by affixing reactive chemical groups to the prosthetic.

There remains a need for a tissue reinforcing prosthetic where the biocompatible component determines the structure of the prosthetic, independently of any underlying fiber or other reinforcement.

### SUMMARY OF THE INVENTION

The present invention encompasses tissue reinforcements in the shape of sheets that are meant to be placed on or between layers of tissue to strengthen one or more layers of tissue. In particular, the tissue reinforcements invention prevent or lessen the extent of a force-induced tissue thinning, bulging and stretching (aneurization).

Traditionally, mesh formed by weaving or bonding fibers together in regular arrays has been used in the repair of tissue herniations. These meshes provide porosity through which tissue grows and thereby anchors the repair. The tissue in-growth is promoted by selecting polymeric materials that induce tissue inflammation, which results in fibrosis. Growth through the mesh is secondary to growth along the surface of the mesh. Over time, the fibrosis builds on both sides of the mesh and becomes many times thicker than the mesh itself.

A natural consequence of fibrotic healing is that the fibrotic tissue is eventually reabsorbed by the body. The loss of fibrotic tissue is mediated by phagocytic cells distributed throughout the tissue volume, and is volume related rather than confined to a surface. As a result, the fibrotic layer formed on the mesh contracts not only in the direction normal to the mesh, but also in the plane of the mesh. The fibrotic layers on each side of the mesh are coupled together by the tissue through-growth in the porosity of the mesh. As the layers contract they pull the mesh with it, causing it to fold and buckle. The result is usually a hard and painful locus of tissue and implant.

In this context, ingrowth is a growth of tissue to or into a fabric, mesh or similar device, connecting an artificial surface to living tissue, but not necessarily extending thought it. A through-growth (also written “through growth” or “throughgrowth”) is a continuous tissue connection extending through the fabric or mesh or other artificial surface from one living tissue to another. The two types of growth may co-exist.

Prosthetics provide opportunities for infection to gain a foothold in the body by protecting microbes from being attacked by the body's natural defenses. Microbes multiply on the prosthetic surface, protected on one side by the prosthetic and on the other side by a protective secretion. Although it is unproven that ingrowth is necessary to anchor the prosthetic, ingrowth is thought by some to provide a benefit by decreasing the likelihood of infection. This may be because ingrowth provides a physical barrier to unchecked proliferation of the microbes, keeping the colony size small enough to eventually be eliminated by the body.

Whether a prosthetic needs to encourage tissue ingrowth by inducing an inflammatory response in the surrounding tissue is unclear. Polyester mesh, which induces far less tissue fibrosis, is not associated with elevated incidence of infection, and the fibrosis is primarily concentrated in the pores of the mesh. Microscopic examination of tissue ingrowth in both polyester and polypropylene mesh suggests it is the porosity of the mesh that promotes through growth, and it is the inflammatory potential of the mesh that promotes fibrosis along the plane of the mesh. It is through growth, and not fibrosis along the plane of the mesh, that prevents microbe proliferation along the surface of the mesh.

It is therefore desirable to promote through growth and discourage microbe proliferation. It is also desirable to discourage fibrosis along the mesh that leads to mesh contraction. Growth of tissue through a prosthetic is largely determined by hole geometry. Hole geometry determines through growth in two ways: 1) hole size moderates the degree to which the opposing layers of tissue come in contact, and 2) hole density moderates the degree of tissue response. Larger hole size provides for more tissue-to-tissue contact, thereby promoting through growth. Since through growth is associated with the release of tissue growth factors, a higher density of holes (or generally, of fibers) promotes fibrosis in the plane of the prosthetic by shortening the diffusion path between cells that have been stimulated to produce such growth factors. This exposes nearby cells to higher levels of growth factors, more easily creating conditions for additional fibrosis to form.

On the other hand, hole size and density also contributes to such other factors as flexibility, elasticity, tensile strength, and roughness of the prosthetic. It is desirable to de-couple these factors from the choice of hole geometry.

For example, it is characteristic of fiber-constructed meshes that their tissue response is inseparably linked with the prosthetic's mechanical properties. To obtain suitable “suture tear-through” strengths, the fibers must be selected to have a particular combination of thickness, tensile strength and chemical composition. For the prosthetic to be suitably flexible, the porosity or fiber density must be below some maximum. Hence, in current practice, chemical composition and pore geometry are largely determined by the desired mechanical properties of the prosthetic.

Hydrogels are uniquely biocompatible. Hydrogels contains large amounts of loosely bound water, and the hydrogel's water is free to equilibrate in osmolarity and chemical composition with the surrounding tissue. This exchange of the hydrogel water with the surrounding tissue water makes prosthetics made from hydrogel more tissue-like and hydrophilic, and discourages the attachment of protein markers on the surface of the prosthetic. These features dramatically reduce the inflammatory potential of the prosthetic.

High water content hydrogels typically possess low tensile strength and no macroscopic porosity. Hydrogels can be given an increased tensile strength by reinforcing the hydrogel with fiber of suitable strength. This fiber can be discrete strands, or woven as a mesh. Non-porous sheets of hydrogel impregnated with fiber reinforcement can be made such that no portion of the fiber is exposed to tissue. Thus the chemical composition of the fiber does not necessarily contribute to a tissue response. Sheets made in this way can be punched or laser drilled with any desired hole geometry without greatly affecting the mechanical properties of the prosthetic. The extent of punching and its pattern may expose cut ends of reinforcing fibers. These ends may serve as useful foci of fibrosis, as described further below.

The present invention encompasses tissue reinforcing prosthetics that promote tissue through growth and discourage tissue growth along the plane of the prosthetic. This is achieved generally by reducing as far as practicable the inflammatory aspects of the prosthetic, and by selecting a porosity with frequency and size suitable for discouraging infection and tissue growth in the plane of the prosthetic. The mechanical properties of the prosthetic are independently adjusted by selecting a fibrous reinforcement having suitable tensile strength and other mechanical properties, and incorporating it at a suitable density.

The present invention describes porosity geometry that can be effectively deployed on any highly biocompatible substrate, absorbable and non-absorbable, and is not limited to hydrogel compositions. In particular, PTFE is a suitable substrate for achieving the objectives of the present invention.

### BRIEF DESCRIPTION OF THE FIGURES

FIG. 1 is a cross-section of a coated mesh of the present invention, and

FIG. 3 is a face view of the same.

FIG. 2 illustrates the prior art.

FIG. 4 shows additional patterns punched into the device of the invention.

FIGS. 5a and 5b show the relative exposed fiber surface areas of prior art materials (FIG. 5a) and the inventive materials (FIG. 5b).

### DETAILED DESCRIPTION OF THE INVENTION

Prepolymers suitable in the present invention form both absorbable and non-absorbable hydrogels. Nonabsorbable hydrogel compositions suitable for the present invention are described in U.S. Pat. No. 6,296,607, in U.S. application Ser. No. 10/020,331, and in copending U.S. application Ser. No. 11/092,396. Absorbable compositions are described in U.S. application Ser. No. 10/651,797. Each of these references is incorporated in its entirety by reference. While these compositions are preferred, there are other types of hydrogels that may be used in the invention. Some of these are described in U.S. Pat. No. 5,410,016 and its references.

### Non-Absorbable Prepolymers

Prepolymers of polyurethanes form the preferred hydrogels of the present invention. They are described in more detail in our copending U.S. application Ser. No. 11/092,396. They are formed by endcapping triols, or triolized diols, with low molecular weight diisocyanate, and then reacting the product of these steps with an excess of water. When the polyol component is a polyalkylene oxide (PAO) containing approximately 75% (70%-95%) ethylene oxide monomers and about 25% (5%-30%) propylene oxide monomers, the resulting hydrogel can contain up to 90% water and achieve desirable stability and strength characteristics. The PAO can be made as a diol (two armed) and later made capable of crosslinking by trimerization with a low molecular weight triol (such as trimethylol propane, TMP) or a higher-functionality material. The PAO can also be made as a tri-armed structure by starting with a trifunctional starter, such as TMP.

Preferred prepolymers are the product of reacting about 20% by weight to about 40% by weight TDI (toluene diisocyanate), 65% by weight to about 85% by weight polyalkyleneoxide (PAO) diol and about 0.5% by weight to about 2% by weight TMP (trimethylol propane). More preferably, the composition is the product of reacting in weight ratios about 20% to about 25% TDI, 70% to about 80% PAO diol and about 0.7% to about 1.2% TMP. Most preferably, the composition is the result of reacting about 23% to about 25% TDI, about 73% to about 77% diol and about 0.7% to about 1.0% TMP. Most preferably, the composition is the result of reacting about 24% TDI, 75% diol and about 0.7% to 1.0% TMP. In all of the above reaction products, the preferred diol is 75% polyethylene glycol and 25% polypropylene glycol, but can have values in the range of about 70%-95% ethylene oxide monomers and 5%-30% propylene oxide monomers. These ranges, themselves somewhat approximate, are discussed in our copending applications US 2003-0135238 and Ser. No. 11/092,396.

Other suitable compositions are the product of reacting about 20% by weight to about 40% by weight IPDI (isophorone di-isocyanate; an aliphatic diisocyanate with a slower reaction rate than TDI), 65% by weight to about 85% by weight diol and about 1% by weight to about 10% by weight TMP. More preferably, the composition is the product of reacting in weight ratios about 25% to about 35% IPDI, 70% to about 80% diol and about 2% to about 8% TMP. Most preferably, the composition is the result of reacting about 25% to about 30% IPDI, about 70% to about 75% diol and about 1% to about 8% TMP. Most preferably, the composition is the result of reacting about 25% IPDI, 70% diol and about 1% to 2% TMP. In all of the above reaction products, the preferred diol is 75% polyethylene glycol and 25% polypropylene glycol.

### Biodegradable Prepolymers

The present invention includes implantable pre-polyurethane compositions that form solids in the body that contain links that are hydrolysable. Degradable materials are described in more detail in our copending application U.S. Ser. No. 10/651,759. The following summary may be supplemented by this or other references.

One type of hydrolysable link is an ester link. These are formed in the polyurethane when the polyol of the pre-polymer has been reacted with an alpha-hydroxy or other hydroxy carboxylic acid. Suitable hydroxy carboxylic acids include butyric, glycolic, lactic and propionic acids, and their cyclic or lactone forms. Other degradable groups include trimethylene carbonate, dioxanone, caprolactone, and anydride bonds.

The esterification process involves heating the acid, or a cyclic lactone form of a hydroxy acid, or other degradable entity with the polyol until the acid and hydroxyl groups form the desired ester links. The higher molecular weight acids are lower in reactivity and may require a catalyst making them less desirable.

Degradability of the formed polymer depends on the type of acid or acid system (multiple acids) used as well as the type of polyol or polyol system used. Common polyols useful in the present invention are aliphatic or substituted aliphatic alcohols containing a minimum of 2 hydroxyl groups per molecular chain. Since a liquid is desired, the polyols are low molecular weight compounds containing less than 8 hydroxyl groups. Alternatively, polyester and polyether polyols or mixtures of these are useful. Generally, hydrophilic polyols or polyol components will accelerate biodegradation by swelling the formed polymer whereas hydrophobic polyols tend to strengthen the formed polymer and delay polymer loss.

Suitable alcohols include, without limitation, adonitol, arabitol, butanediol, 1,2,3-butanetriol, dipentaerythritol, dulcitol, erythritol, glycerol, hexanediol, iditol, mannitol, pentaerythritol, sorbitol, sucrose, triethanolamine, trimethylolethane, trimethylolpropane, and combinations of ethylene and propylene oxides initiated by polyols or by various amines.

Polyether polyols suitable in the present invention are readily available and include random copolymers, block copolymers, and graft copolymers, as well as polyether polyols of different monomer compositions linked together by chain extending reagents, such as diisocyanates. Triols of polyester and polyether may be used provided they are in liquid form, normally less than 8000 MW. Degradation of the formed polymer can be controlled by mixing the hydroxy ester with any of the above polyols.

A preferred polyol composition includes a trifunctional hydroxy acid ester and linear polyoxyethylene glycol system. In the prepolymer, the ester acts as the crosslinking agent linking together the polyoxyethylene glycol. In the formed polymer, chemical action degrades the ester leaving essentially linear chains that are free to dissolve or degrade. Interestingly, in this system, increasing the percentage of degradable crosslinker increases rigidity, swell and solvation resistance in the formed polymer.

Other polyol systems include hydroxy acid esterified linear polyether and polyester polyols optionally blended with a low molecular weight alcohol. Similarly, polyester and polyether triols esterified with hydroxy acid are useful.

Other polyol systems include the use of triol forming components such as trimethylolpropane to form polyols having three arms of linear polyether chains.

The prepolymer of the present invention is formed by capping the polyols with polyisocyanate, preferably a diisocyanate. However, suitable isocyanates have the form

R(NCO)ₓ

where x is 2 to 4 and R is an organic group.

Another approach is to graft the polyol onto a biodegradable center. Suitable polymers for inclusion as center molecules are described in, for example, U.S. Pat. No. 4,838,267. They include alkylene oxalates, dioxepanone, epsilon-caprolactone, glycolide, glycolic acid, lactide, lactic acid, p-dioxanone, trimethylene carbonate, trimethylene dimethylene carbonate and combinations of these. The center molecule may be a chain, a branched structure, or a star structure. Suitable star structures are described in, for example, U.S. Pat. No. 5,578,662. Isocyanate capped alkylene oxide can be reacted with these molecules to form one or more extended chains.

Center molecules such as those listed above may form rigid solids upon polymerization. Therefore, it is generally more useful to ensure at least 80% alkylene oxide is in the final polymerized structure. Furthermore, the alkylene oxide should be comprised of at least 70% ethylene oxide.

### Anti-Adhesion Prepolymers

Edlich et al in the Journal of Surgical Research, v. 14, n. 4, April 1973, pp 277-284 describes the results of applying a topical solution of 10% ethylene oxide/propylene oxide copolymer to wounds. Reduced inflammatory response at the wound was found for copolymer solutions containing ethylene oxide:propylene oxide in the ratio of 4:1. Inflammation is known to be associated with adhesion formation around surgical sites.

The polymer of the present invention is preferably comprised of an isocyanate capped and subsequently crosslinked structure of poly(ethylene oxide/propylene oxide) (PEOPO; also known as a “poloxamer”). Under biodegradation or absorption of the in situ formed polymer, essentially whole chains of PEOPO are released into the body. The decomposition of the implant provides for a continuous supply of PEOPO which can serve as an anti-adhesion agent during wound healing. Since polyoxyalkylene block copolymers are absorbed by tissues, the degradation products are eventually excreted in a non-metabolized form. We use the word “poloxamer” to mean any copolymer of ethylene oxide and propylene oxide, whether random, block, or graft, and with either EO or PO groups on the end, and optionally containing small amounts of other oxiranes or similar monomers. A key attribute of poloxamers is their possession of both hydrophilic and hydrophobic monomers, and their corresponding tendency to segregate portions of their molecular chains to particular environments.

Further increases in the rate of release of PEOPO can be made by adding un-derivatized PEOPO directly to the prepolymer of this invention. The result is a prepolymer which will spatially trap PEOPO as a hydrogel.

The three dimensional structure of the crosslinked implant holds the PEOPO hydrogel by physical quasi or pseudo crosslinks, typically ionic or hydrogen bonds. Since these bonds are reversible, thermodynamic considerations will drive the PEOPO to slowly elute from the implant. This action will decrease the volume of the implant, without breaking the bonds of the crosslinked structures. Thus, an absorbable implant is formed having potentially both absorption and decomposition pathways to volume loss.

### Reinforcement Fibers

The present invention describes sheets made by entirely encapsulating fibrous reinforcement material with a biocompatible, permanent solid, or substantially encapsulating reinforcement materials with said solid. In some cases, some of the reinforcement material may be exposed during hole punching or other procedures, and will be exposed to tissue when implanted at locations intended to stimulate tissue proliferation.

Suitable reinforcement materials are fibrous in nature, and include mesh materials and fabric materials. Commercial mesh materials include brands such as SurgiPro (Tyco) and GyneMesh (J&J) as well as other widely available surgical meshes. The present invention, in a preferred embodiment, differs from previous coated meshes in that the porosity of the mesh is not necessarily retained in the present invention, and that the geometry of the surface presented to tissue is predominantly determined by the hydrogel component of the prosthetic. In the invention, both fabrics and meshes may be non-woven or woven; woven materials may include knitted materials and other materials in which fibers are periodically joined together in the making of a fabric.

Other suitable fibrous reinforcements include flock of various dimensions and compositions and spin bonded or adhesive bonded sheets of fibrous materials. In particular, fibrous elements composed of polypropylene are preferred for their strength, light weight and biocompatibility. A currently preferred fabric is a knitted polyester producing a pattern of hexagonal openings.

Other approaches to providing a reinforced hydrogel comprise providing a solid polymeric sheet, coating it with a hydrogel or hydrogel precursor, and punching an appropriate pattern into the composite. Another approach is to produce a mesh or a preformed porous material, and coat it with a solution of prepolymer in organic solvent, followed by drying the composite, and optionally but preferably allowing the composite to cure by the action of atmospheric moisture.

The polymeric hydrogel layer resulting from coating a mesh or sheet with liquid prepolymer, optionally deposited from an organic solvent and dried, depends both on the chemistry of the prepolymer, its molecular weight, and on the method of reaction the polymer to produce the coating. This will be described in more detail below.

### Techniques for Constructing a Tissue Reinforcement Prosthetic

The present invention is a prosthetic characterized in having mechanical properties that are independent from its biocompatibility and tissue response properties. There are two types of prosthetics that satisfy the goals of the present invention. One type is those prosthetics made of a single, biocompatible material onto which perforations, surface texture and the like provide for the tissue response. The second type is prosthetics in which the prosthetic's mechanical properties are derived from a reinforcement material, possibly with undesirable tissue response properties, and the reinforcement material is encapsulated by a material which will form a hydrogel in the presence of bodily fluids to an extent sufficient to remove or lessen any undesirable tissue response.

It is noteworthy that the selection of the geometry of the through-growth holes made in the prosthetic is not, in the present invention, restricted by the reinforcement element. An alternate description is that the prosthetics of the invention are formed from two or more materials, wherein at least one of the materials is a hydrogel or a substance that becomes a hydrogel when placed in the body, and another component is a non-hydrogel solid.

The prepolymers described previously form crosslinked solid hydrogels when activated by contact with water. The reinforcing element of the prosthetic is typically combined with the prepolymer, or with a water activated solution of prepolymer, before polymerization is complete.

For example, polypropylene flock may be mixed with and suspended in a prepolymer, and the mixture is then allowed to air cure in a vessel that imparts a shape to the mixture. Air cure refers to the slow polymerization of the prepolymer that occurs due to water vapor in the air. The mixing may be facilitated by the addition of an organic solvent such as acetone or toluene, and the solvent then evaporated by heating the mixture in the curing vessel, followed by exposure to water vapor.

Similarly, prepolymer may be placed in a curing vessel and a fibrous matrix such as a cloth or mesh is then placed in the vessel so that prepolymer encapsulates the fibrous matrix. Cure can again be via water vapor.

The prepolymer may be mixed with a quantity of water, or saline, to achieve a desired degree of hydration in the polymerized hydrogel. Some prepolymers, such as those prepared with toluene diisocyanate, are fast reacting, typically on the order of tens of seconds. When the prepolymer is fast reacting, the prepolymer may be mixed with water at the point of application to flock or mesh. For example, two intersecting jets of prepolymer and water may be applied to a curing vessel, mold, or fibrous matrix. Alternatively, the prepolymer and water may be mixed at reduced temperature, typically just above freezing, to decrease the reaction rate. Such reduced temperature preparations can have a useful pot life ranging from several minutes to hours.

Fibrous matrix saturated with a preparation of water and prepolymer may be passed between roller to obtain a desired surface finish or prosthetic thickness. Similarly, the saturated fibrous matrix may be pressed between plates.

Hydrogel components that contain a high percentage of water may swell in the body. Even small amounts of swelling, when combined with a non-swelling fibrous reinforcement, can cause buckling of the prosthetic. To eliminate this undesirable condition, the prepolymer may be combined with polyol and water to form a polyol-polyurea hydrogel. (Note that the reaction rate of the polyol hydroxyl group with the isocyanate is typically an order of magnitude slower than the reaction of isocyanate with water.) For example, the polyol may be polyethylene oxide, or alternatively the polyol may be the same polyol used to construct the prepolymer. Alternatively, the swelling preventer may be a pharmaceutical cream such as Emulgade 1000 NI (Cognis), a mixture of cetearyl alcohol and ceteareth-20.

In general, the equilibrium water content of the coated fibrous materials depends on the nature of the coating and the manner of its application. When the reactive isocyanate group on the poloxamer is the residue of a highly reactive diisocyanate such as toluene diisocyanate, and the molecular weight of the triolic (three armed) or triolized (diol precursors joined to a small triol) poloxamer is low, for example below about 4000 daltons, then the coating formed by “air curing” is dense, and the amount of water absorbed when exposed to liquid water or body fluids is small, for example about 4% of the weight of the coating. The weight of a dry coated material is usually about half coating and half fibrous material, but the detailed breakdown depends on the nature of the fibrous material, especially its fiber diameter. Although 4% seems small, it is sufficient to allow the coating layer to be hydrophilic, tissue compatible, and non-fibrotic. In general, living cells do not adhere well to such coatings.

When the TDI-prepolymer molecular weight is higher, then more water can be absorbed by an air-cured coating, for example 50 to 100% by weight. When the diisocyanate used to cap the polymer is a slow reacting isocyanate, such as diisophorone diisocyanate, then swelling of the air cured polymer layer is typically about 30% or more below 4000 daltons of molecular weight of the prepolymer, and 50%-150% at higher weights.

Coating of a substrate with an activated prepolymer, for example a prepolymer that has just been mixed with water or buffer, is an alternative method of coating. It is especially suited to a method in which holes are to be punched later, either before or after drying a layer of gel formed in situ.

Sheets prepared by any of the methods described typically need to be perforated to allow beneficial tissue through growth. Through growth provides long-term fixation of the prosthetic and mitigates against infection, as described above. In some cases, with careful selection of the fibrous mesh, a material can be produced directly by coating and air curing that has an appropriate degree of through growth.

In most surgical uses of a tissue reinforcement prosthetic, the prosthetic is shaped by cutting to facilitate prosthetic integration to the surgical repair site. It is preferred that the hole pattern not constrain the surgeon, and thus a regular or repeating pattern is preferred. The density of holes in the pattern must be selected to encourage through growth sufficient to discourage microbial proliferation. On the other hand, the hole density should not be so great as to encourage fibrotic encapsulation in the plane of the prosthetic.

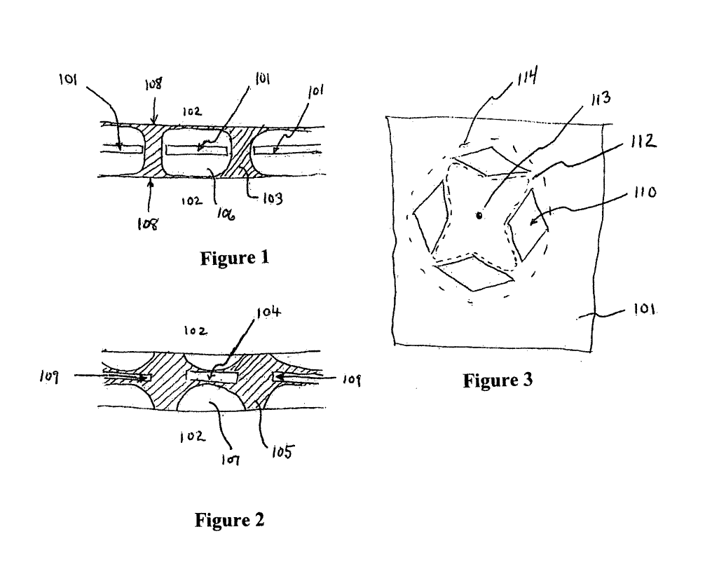

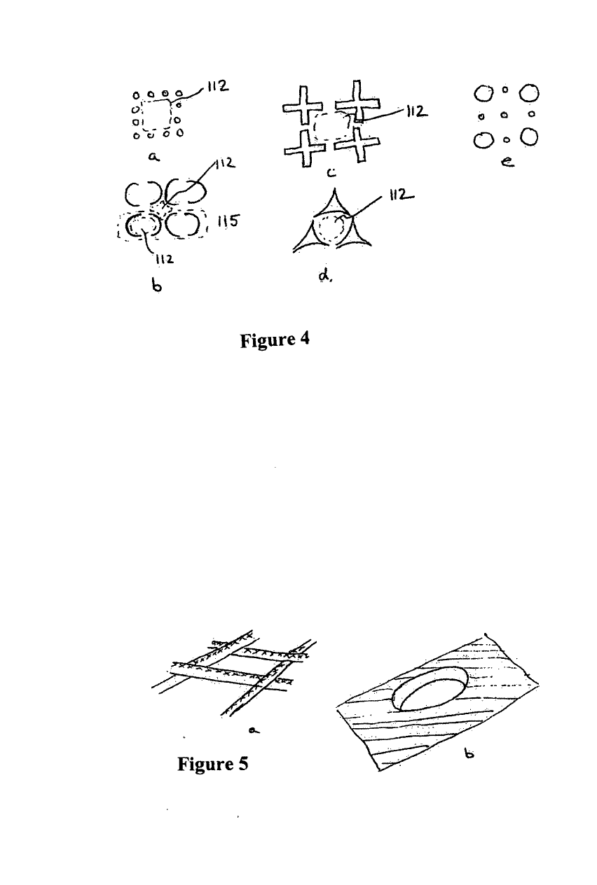

Referring now to FIG. 1, a cross sectional view of a prosthetic sheet 101 sandwiched between two layers of tissue 102 with tissue through growth 103 is depicted. FIG. 3 shows a face-on view of the sheet 101. The geometry of the through growth shown is representative of a prosthetic sheet 101 which discourages tissue growth on its surface. Such a condition would be encountered if the prosthetic were made of a hydrogel, or was coated with a hydrogel. Referring now to FIG. 2, a similar arrangement of prosthetic 104 and tissue 102 is depicted, with tissue through growth 105, but where prosthetic 104 is comprised of a tissue ingrowth promoting substance such as polypropylene, and is not hydrogel coated.

The tissue ingrowth 105 in FIG. 2 is geometrically different from the through growth 103 depicted in FIG. 1. The tissue growth is also different in several functional aspects. For instance, void 106 in FIG. 1 remains fluid filled allowing the prosthetic 101 to remain loosely coupled to the tissue, whereas void 107 in FIG. 2 fills with fibrotic tissue over time, and rigidly couples prosthetic 104 to the surrounding tissue 102. The mass of fibrotic tissue developed as a result of these differences will be much greater in the situation shown in FIG. 2 when compared with that shown in FIG. 1. Moreover, as the tissue heals, the fibrotic tissue loses mass and contracts. In FIG. 1, the contraction is primarily along arrows 108, perpendicular to the mech, bringing the two tissue layers 102 together without introducing stress in the plane of the prosthetic 101. In FIG. 2, the contraction is primarily along arrows 109, in the local plane of the prostheric, causing the prosthetic 104 to bunch relative to the tissue layers 102.

FIG. 3 depicts the prosthetic 101 of FIG. 1 in plan view. The prosthetic 101 contains perforations 10 which provide for through growth like that shown as 103 in FIG. 1. The four perforations shown comprise a “perforation set” 114, which can be repeated across the surface of the prosthetic 101. The dashed line 112 represents the perimeter of a space corresponding to void 106 of FIG. 1, illustrating the compartmentalization of the prosthetic 101 surface, a feature known to be important in preventing microbial proliferation.

Generally, the amount of fibrotic tissue is a function of the separation distance between tissue planes. Accordingly, thinner prosthetics are preferred to thicker ones with the same mechanical properties. Larger holes allow tissue to fill the void and come in closer contact. Holes with chamfered edges accomplish the same end without increasing the open area in the prosthetic.

Referring again to FIG. 3, a preferred prosthetic 101 would promote tissue compartmentalization 112 of the prosthetic 101, with a compartment area less than about 25 mm². Additionally, a non-fibrotic anti-microbial such as metallic silver may be incorporated within each compartment 112, at approximately the center 113. Preferably the perforation set 114 is repeated across the surface of the prosthetic 101 so as to not violate the 25 mm² limit on any one compartment 112.

The shape of the holes are preferably not circular so as to establish the largest area for region 112 while minimizing the cross sectional area of the perforations. Examples of other preferred patterns are illustrated in FIG. 4. Small circular holes are acceptable if they describe a large region 112, as in FIG. 4a. A hole pattern may establish a plurality of regions 112 as illustrated in FIG. 4b. Additionally, a regular pattern establishing a row 115, may be staggered with respect to an adjacent row 115. Holes may serve a purpose other than to establish compartmentalization. For example, small holes may be provided along with large holes, where the small holes are more frequent and provide for vascular penetration and the large holes provide for compartmentalization, as shown in FIG. 4e. More complex shapes can be used, such as those shown in FIGS. 4c and 4d.

Prosthetics promote microbial survival because they essentially shield the microbe from detection or attack on at least one side, and provide an anchoring site from which the microbe can proliferate. Microbial proliferation is enhanced further in prosthetics which contain voids that are not in intimate contact with perfused tissue. For example, as shown in FIG. 5a, a traditional woven mesh possesses a porosity and thickness which prevents tissue contact with half its surface area. Only the areas cross hatched (and their equivalents on the opposite face) are in contact with tissue. The “sides” of the fibrils are exposed and can stimulate fibrosis. For this reason, historically successful meshes are highly inflammatory and develop a chronic fibrotic response, so that tissue quickly fills the non-contacting areas.

A similar situation occurs with perforations in a sheet, as shown in FIG. 5b, where the walls of the hole are not in tissue contact. The advantage of perforated sheet over woven mesh is that the amount of surface area not in contact with tissue can be controlled by hole size and density. This is less easily accomplished with mesh, where the fiber density and thickness determines the mechanical properties of the mesh.

However, it is one aspect of the present invention to use an inflammatory reinforcement fiber such as polypropylene. In this case, when the holes are punched into the hydrogel sheet, the fibers are exposed at the edges of the holes, promoting a local inflammatory response. Since the polypropylene exposure is localized to the holes, it does not promote fibrosis across the plane of the prosthetic, and consequently does not promote prosthetic contraction. However, it does promote through growth and compartmentalization of the prosthetic surface.

### EXAMPLES

Below are described specific embodiments of the present invention.

### Example A

### Non-Absorbable Prepolymer

Seven hundred grams of Diol UCON 75-H-1400 (Dow Chemical), a poloxamer with about 25% PO and 75% EO subunits, are heated to 49 deg. C. and stirred under a continuous flow of argon for 24 hours. The prepared diol is cooled to room temperature (22 deg. C.) and 113.40 g of Toluene Diisocyanate added. The mixture is stirred under an argon blanket and the temperature of the solution is increased linearly to 60+/−2 deg. C. over a two hour period. The mixture is maintained at these conditions until the concentration of NCO (isocyanate) drops to 2.95%. When this target is reached, 6.26 g of Trimethylolpropane is added, and the mixture stirred under argon at 60+/−2 deg. C. until the % NCO reaches 2.21. This finished prepolymer is cooled under argon, and stored in a dessicator and away from light.

### Example B

### Hydrogel Composition

A hydrogel useful for forming sheets of fiber reinforced prosthetic is obtained by mixing at room temperature equal parts by volume Example A, from UCON 75-H-1400, and isotonic saline.

### Example C

### Hydrogel Composition

A hydrogel useful for forming sheets of fiber reinforced prosthetic is obtained by mixing at room temperature equal parts by volume of the prepolymer of Example A, and Emulgade 1000 NI (Cognis).

### Example D

### Prosthetic

Prosthetics were formed by applying the mixtures of Example B or C, before curing, to SurgiPro mesh (Tyco). After the hydrogel cured, diamond shaped holes as shown in FIG. 3 were punched into the hydrogel/mesh composite. The whole set was comprised of 2 mm by 1 mm holes spaced 2 mm from the center 113 of the hole set. The hole sets were distributed regularly across the prosthetic surface on 4 mm spaced centers.

### Example E

### Prosthetic

Prosthetics were formed by applying the mixtures of Example B or C, before curing, to GyneMesh (J&J), After the hydrogel cured, diamond shaped holes as shown in FIG. 3 were punched into the hydrogel/mesh composite. The whole set was comprised of 2 mm by 1 mm holes spaced 2 mm from the center 113 of the hole set. The hole sets were distributed regularly across the prosthetic surface on 4 mm spaced centers.

### Example F

### Prosthetic

Prosthetics were formed by applying the mixtures of Example B or C, before curing, to Mpathy mesh (Secant), After the hydrogel cured, diamond shaped holes as shown in FIG. 3 were punched into the hydrogel/mesh composite. The whole set was comprised of 2 mm by 1 mm holes spaced 2 mm from the center 113 of the hole set. The hole sets were distributed regularly across the prosthetic surface on 4 mm spaced centers.

### Example G

### Prosthetic

Prosthetics were formed by applying the mixtures of Example B or C, before curing, to commercially available 100 micron thick spun bound polypropylene sheet, After the hydrogel cured, diamond shaped holes as shown in FIG. 3 were punched into the hydrogel/mesh composite. The whole set was comprised of 2 mm by 1 mm holes spaced 2 mm from the center 113 of the hole set. The hole sets were distributed regularly across the prosthetic surface on 4 mm spaced centers.

### Example H

### Implantation

The composition of Example B was applied to SurgiPro mesh as in Example C. The cured composite material was not punched. It was implanted subcutaneously in the back of a rat and removed after 2 weeks. Uncoated SurgiPro mesh was implanted in the same rat as a control, and likewise removed at 2 weeks. On gross observation, there was more fibrosis on the uncoated (control) mesh.

### Example I

### Dry Coating Implant

The prepolymer of example A was mixed with an equal volume of acetone. The mixture was sprayed on a piece of SurgiPro mesh, sufficiently to produce a layer visually estimated to be about 100 micron thick (before drying), and allowed to air dry. The air dried material was implanted as in Example H, and likewise removed at 2 weeks. The coated mesh was observed to have less fibrosis than the uncoated mesh.

The tissue reinforcers of the invention can be used for any medical condition in which a repair mesh or similar device is currently used, or contemplated. Uses include, without limitation, treatment of a hernia or herniation, whether of a specific site or caused by injury; the repair of any aneurysm or prolapse; and the treatment of specific forms of these disorders, including, without limitation, treatment of rectocele, cystocele, enterocele, enterocystocele, prolapse of the uterus, inguinal hernia, or traumatic wound. Coated meshes of the invention may be used postoperatively in many surgical procedures to provide local strength during the healing process.

Many other examples of the invention will be suggested to a skilled person by the description and the figures. The invention is not limited by the description or examples provided, but rather by the claims.

1. An improved implantable prosthetic reinforcement device for tissue, the device comprising:
a structural component providing reinforcement;
a hydrophilic surface component providing tissue compatibility; and
through-holes passing through the device and being of sufficient size to allow through-growth of the tissue that is being reinforced;
wherein the spacing of the through-holes is selected to minimize fibrotic stimulation.

2. The device of claim 1, wherein the hydrophilic surface component comprises an isocyanate-terminated crosslinkable poloxamer containing at least about 70% ethylene oxide monomer by number.

3. The device of claim 1 wherein the structural component is selected from a woven fabric or mesh, a non-woven fabric or mesh, a knitted fabric or mesh, an embedded dispersed fibrillar component in the surface component, and a perforated or non-perforated sheet of polymeric material.

4. The device of claim 1 wherein the through-holes are made after the combination of the hydrophilic surface component and the structural component.

5. The device of claim 1 wherein the through-holes are made before the combination of the hydrophilic surface component and the structural component.

6. The device of claim 1 wherein the through-holes are formed in a repeating pattern in the device.

7. The device of claim 6 wherein the pattern is selected to provide sufficient compartmentalization of the device along its planar dimensions to restrict the growth of microbial colonies.

8. The device of claim 6 further providing a portion of an antimicrobial material disposed in each repeat or compartment of the pattern of the device.

9. The device of claim 6 wherein the area of the repeating pattern is less than about 25 square millimeters.

10. The device of claim 1 wherein the hydrophilic surface component is applied to the structural component by spraying.

11. The device of claim 10 wherein the surface component is applied in an organic solvent which is removed by drying.

12. The device of claim 1 wherein the hydrophilic surface component is applied to the structural component by coating a liquid hydrophilic surface component onto a preformed structural component.

13. The device of claim 1 wherein the hydrophilic surface component and the structural component are mixed and then spread out to form a film.

14. The device of claim 1 wherein the wherein the hydrophilic surface component is partially cured during or after its application to the structural component by admixture with a compound promoting curing of the hydrophilic surface component.

15. The device of claim 14 wherein the compound promoting curing comprises one or more of an aqueous solution and a polyol.

16. The device of claim 14 wherein the compound promoting curing comprises atmospheric moisture.

17. Use of the device of claim 1 for the treatment of one or more of a hernia or herniation of an internal organ; an aneurysm or prolapse of an internal organ; or of a rectocele, enterocele, cystocele, enterocystocele, or traumatic wound.

18. A method for the minimization of fibrosis-induced contracture in a reinforcing prosthetic, the method comprising:
providing, as the outermost layer of said reinforcing prosthetic, a hydrophilic material, consisting essentially of a poloxamer-based polyurethane, wherein said hydrophilic material hydrates to a water content of at least about 4% upon contact with aqueous solutions or fluids.

19. The method of claim 18 wherein the polyurethane is cured in situ after its application to the reinforcing component.

20. The method of claim 18 further comprising the step of providing through-holes formed in a repeating pattern in the device, wherein the area of a repeat of a through-hole pattern is less than about 25 square millimeters.
